(19) United States
(12) Patent Application Publication
(10) Pub. No.: US 2008/0213232 A1
(43) Pub. Date: Sep. 4, 2008

(54) Radial Glial Cells Promote Nerve Regeneration and Functional Recovery Following Spinal Cord Injury

(75) Inventor: Martin Grumet, New York, NY (US)

Correspondence Address: HOFFMANN & BARON, LLP, 6900 JERICHO TURNPIKE, SYOSSET, NY, 11791, US

(73) Assignee: RUTGERS, THE STATE UNIVERSITY OF NEW JERSEY, New Brunswick, NJ (US)

(21) Appl. No.: 11/665,703

(22) PCT Filed: Oct. 24, 2005
(86) PCT No.: PCT/US2005/038659

§ 371 Date: Mar. 3, 2008

Related U.S. Application Data
(60) Provisional application No. 60/621,841, filed on Oct. 25, 2004.

(51) Int. Cl. A61K 45/00; C12N 5/08; C07K 16/00; A61P 25/00

(52) U.S. Cl. 424/93.21; 435/368; 530/388.1; 530/387.9

(57) ABSTRACT

An enriched population of radial glial cells has been isolated. These cells are nestin⁺, BLBP⁺, and non-tumorigenic. Furthermore, they are capable of exhibiting bipolar morphology, migrating and self-organizing in white matter, and supporting neuronal migration. These cells can be maintained by regeneration in culture. Therapeutic methods of use of these cells are also described.

This invention was made with Government support under Grant No. RO1 NS38112, awarded by the NIH. Therefore, the Government has certain rights in this invention.

### FIELD OF THE INVENTION

The present invention relates to lineage restricted glial precursors from the central nervous system (CNS). More particularly, this invention relates to an enriched population of radial glial cells and its use to promote neuronal regeneration in vivo.

### BACKGROUND OF THE INVENTION

Important advances have been made in understanding conditions that can influence survival and regrowth of neurons following injury to mature CNS tissue. These advances include neurotrophic factors to promote survival and growth of neurons, embryonic neural tissues to provide cellular scaffolds to facilitate neuronal regrowth, and neutralization of inhibitors¹⁻³.

Another approach to repair neural damage is replacement of lost cells, in particular lost neurons and other cells. The expanding possibilities to isolate and differentiate neural stem cells and neural restricted precursors has opened new opportunities for generating different types of neurons for replacement therapies^{4,5}. These approaches are well suited for problems like Parkinson's Disease where replacement of lost dopaminergic cells can yield significant recovery of function⁶.

However, many other neurons, for example those that are damaged in spinal cord injury, have their cell bodies in locations (e.g. cerebral cortex) that are quite far from the site of injury in the spinal cord and therefore are difficult to replace. Thus, there is a need for other strategies to protect neurons and promote their regrowth across sites of injury.

Radial glial cells are a ubiquitous cell type in the developing central nervous system (CNS) of vertebrates. Such cells appear transiently during development spanning CNS tissues where they serve as scaffolds to support migration of neurons⁷ and their processes^{8,9}. Experimental studies have provided convincing evidence that most or all embryonic radial glia initially are neural stem cells (NSC), or are restricted precursors derived from them that give rise to neurons early during embryogenesis and to glia at later stages¹⁰⁻¹².

Recent observations made with a highly polarized rat cell line called C6-R suggested that radial glial-like cells are able to migrate extensively in white matter and infiltrate into lesioned neural tissues^{13,14}. Like radial glia¹⁵, C6-R cells promoted migration of neurons and neuronal processes both in vitro and in vivo^{13,16,17}. However, C6-R, which was derived from C6 glioma, formed tumors when transplanted into the contused spinal cord^{14,46}

Hatten and colleagues have pioneered methods to isolate and study cerebellar radial glia in vitro. However, these cells are highly unstable, loosing their bipolar morphology in the absence of neuronal contact in vitro and in the mature nervous system in vivo¹⁸.

Therefore, there is a need in the art to isolate and identify other populations of radial glial or radial-glial-like cells, which may be useful in repairing neural damage. Desirably, such cells would maintain properties common to radial glial cells for a sufficient period of time to promote cell survival in the injured CNS and regrowth of neurons across sites of injury. Moreover, it would be desirable if these cells lacked tumorigenicity.

### SUMMARY OF THE INVENTION

The present invention provides an enriched population of radial glial cells. These cells have the following characteristics: nestin⁺, BLBP⁺ and non-tumorigenic. The cells are also capable of: exhibiting bipolar morphology, self-renewing, migrating and self-organizing in white matter, and supporting neuronal migration. In some embodiments, the cells are GLAST⁺, or derived from a cell that is GLAST⁺.

Further provided is a method for isolating a radial glial cell population. This method includes providing a mixed population of stem cells; binding the cells to an antibody that recognizes an extracellular portion of GLAST; and isolating the antibody-bound cells from unbound cells.

The present invention also provides a monoclonal antibody that specifically recognizes an extracellular portion of GLAST. This monoclonal antibody may be used to isolate radial glial cells of the present invention.

Further provided herein are therapeutic methods useful for protecting neural tissue from injury and/or for promoting regrowth of injured axons in patients in need thereof. These methods include administering to the patient an enriched population of radial glial cells according to the present invention.

### BRIEF DESCRIPTION OF THE DRAWINGS

FIG. 1 is a comparison of differentiation between one embodiment of a cell population of the present invention (RG3.6 cells) and NSCs in normal spinal cords of different rats at four weeks after cell transplantation. A. is a graph comparing GFP fluorescence with staining for nestin (a marker for non-differentiation); B. is a graph comparing GFP fluorescence with staining for GFAP (an astrocyte marker, indicating differentiation).

FIG. 2A-C are micrographs showing GFP fluorescence of fibroblasts (FB) or RG3.6 cells at 6 weeks after injection of these cells into the spinal cord injury site. D. is a graph of the BBB score observed at different time points after injection of RG3.6 cells or fibroblasts at the spinal cord injury site in different rats.

FIG. 3 shows images of whole mounts of spinal cords injected with medium (A) or RG3.6 cells (B) visualized by light. (C) is a graph comparing the BBB score observed at different time points after injection of RG3.6 cells or medium at the spinal cord injury site in different rats.

FIG. 4 presents graphs comparing deposition of the chondroitin sulfate proteoglycans CS56 (A and B) and NG2 (C and D) in RG3.6 treated rats as compared to the control medium and fibroblast (FB) treated groups.

FIG. 5 presents graphs comparing the dorsal (A and B) and ventral (C and D) neurofilament (NF) protein+areas in injured spinal cords of different rats implanted with RG3.6 cells, as compared to implants of fibroblasts or medium. The results are shown at 6 weeks after spinal cord injury.

FIG. 6 shows fluorescence activated cell sorting (FACS) of embryonic stem cell-derived neural stem cells. A. shows the generation of neural stem cells from embryonic stem cells, and B. shows the isolation of neural stem cells by FACS.

FIG. 7 shows immunostaining of embryonic day (E) 15 cortical cells (A-C), E12.5 rat forebrain sections (D, E), and E15 acute cortical cells (F) with anti-GLAST monoclonal antibody.

### DETAILED DESCRIPTION OF THE PREFERRED EMBODIMENTS

Transplantation has been used to restore function to cells and tissues that have a limited capacity for regeneration. It has become increasingly clear that non-neuronal cells can contribute to creating a more favorable environment in vivo for neuronal regeneration. The present invention is directed to an enriched radial glial cell population and its uses to protect neural tissue and promote axonal regrowth in vivo.

By the terms “enriched”, “enrich”, and the like, it is meant to increase the number and proportion of the inventive cells in a cell sample relative to the number and proportion of other cells. The term is intended to encompass the terms “isolated”, “isolate”, and the like, in which a population of the inventive cells is separated from contaminating cells.

### Cells

The cells of the present invention are characterized by the presence (+) of markers associated with specific antigenic sites identified by antibodies. The inventive cells are also characterized by the absence (−) of certain markers as identified by the lack of binding of certain antibodies. The markers described herein are known in the art. The inventive cells are nestin⁺, BLBP⁺ and non-tumorigenic. Moreover, these cells are capable of exhibiting bipolar morphology, self-renewing, migrating and self-organizing in white matter, and supporting neuronal migration.

In some embodiments, the cells of this invention are GLAST⁺ or derived from a cell that is GLAST⁺. GLAST is a marker characteristic of radial glia. Furthermore, in some embodiments, the cells are negative for the neuron-specific marker β-III tubulin (TuJ1).

Moreover, in some embodiments the cells are SSEA4⁻. SSEA4 recognizes an antigen expressed on mammalian and human embryonic stem cells, but not on neural stem cells or radial glia.

As described in the Examples below, as compared to neural stem cells (NSCs), the cells of the present invention are inhibited from differentiating into neurons, oligodendrocytes and astrocytes in vitro. The present inventor has found that radial glia can be modified so as to retard their differentiation, thus allowing these cells to persist as nestin⁺ elongated cells for extended periods of time in vivo. This characteristic is beneficial for neuronal survival, regrowth and regeneration.

Neuronal regrowth refers to the extension of neuronal processes (axons or dendrites). Neuronal regeneration is a form of neuronal regrowth where neuronal processes regrow across an injury site and form synaptic connections similar to those that had existed before injury. Neuronal regeneration can lead to restoration of function. Neuronal regrowth can also lead to restoration of function, but may do so by utilizing unusual neural connectivity and circuits.

The present inventor has found that introduction of v-myc into radial glia retards differentiation, and promotes neuronal regrowth and regeneration. Therefore, in some embodiments, the inventive cells have been modified to express an oncogene.

It is also within the contemplation of the present invention that other methods may be used to initiate immortalization. For example, in some embodiments, the cells of the present invention have increased expression levels of the Notch protein or an intracellular derivative thereof. In other embodiments, the cells of the present invention have increased expression levels of the BLBP protein. In still other embodiments, the cells of the present invention have increased expression levels of glial growth factor erbB2/receptor. The glial growth factor erbB2/receptor may be activated.

The cells of this invention should either express, or should be modified to express high levels of Notch, BLBP, or glial growth factor erbB2/receptor. For example, the cells may by genetically transduced by any means known in the art, such as DNA transfection, to express these high levels. Any eukaryotic expression vector known in the art which contains at least one expression control sequence that is operatively linked to the DNA sequence or fragment to be expressed would be suitable. Suitable eukaryotic expression vectors are described below.

As shown in the examples below, the cells of this invention differentiate more slowly in vitro than NSCs. However, the inventive cells are capable of being induced to differentiate in vitro into oligodendrocytes identified by expression of galactocerebroside, (GalC), and astrocytes identified by expression of GFAP, an astrocyte-specific cytoskeletal protein.

In fact, as discussed in further detail below, it may be desirable to eventually induce at least some of the radial glial cells of this invention to differentiate into oligodendrocytes. The genes olig1 and olig2 are critical for differentiation into oligodendrocytes. Oligodendrocytes function to produce central nervous system myelin, which insulates neuronal axons. The production of myelin would allow signals to be transmitted more efficiently.

The cells of the present invention may be either TuJ1⁺ or TuJ1⁻. The cells express the neuron-specific marker TuJ1 after differentiation in vitro. This characteristic is unlike previously described A2B5⁺ E-NCAM⁻ glial restricted precursor (GRP) cells, which can be clonally expanded and induced to differentiate in vitro into oligodendrocytes, astrocytes, but not into neurons (see, for example U.S. Patent Application Publication No. US 2003/0109041A1). Moreover, unlike GRP cells, in some embodiments, the cells of the present invention are A2B5⁻. A2B5 is a monoclonal antibody against a glycolipid and is used to isolate GRP cells.

The present invention is illustrated using RG3.6 cells isolated from rat. The invention, however, encompasses all isolated mammalian radial glial cell populations having the characteristics described herein and is not limited to the RG3.6 clone, nor to radial glial cells from rat. Mammalian radial glial cells can be isolated from human and non-human primates, in addition to rats.

Several cellular markers have been used to identify radial glia in vivo and in vitro. Markers of radial glia that are shared with NSC include nestin³⁹, which is also found in humans³⁴. Markers of radial glia that are shared with astrocytes include brain lipid-binding protein (BLBP)³⁸, and GLAST⁴⁴. BLBP and GLAST are detectable in human radial glia. For example, see references 45, 32 and 40. Human glast^{32,40} is highly conserved with other mammalian Glast proteins. Other markers that are useful for identifying radial glia include A2B5³⁷, which recognizes a carbohydrate epitope that has been used to define glial restricted precursors⁴², including human cells³⁵. The A2B5 marker is expressed on some radial glia⁴¹. TuJ1³³ has been used to identify neurons in various mammals, including humans⁴³. SSEA4³⁶ recognizes an antigen expressed on mammalian and human embryonic stem cells, but not on neural stem cells or radial glia. Therefore, SSEA4 can be used to distinguish radial glia from embryonic stem cells.

The cells of the present invention may be isolated from the central nervous system of a mammal. In some embodiments, the cells are derived from neurospheres. For example, as described in the Examples, the RG3.6 cells were derived from neurospheres that were generated from rat embryo forebrains. Neurospheres are free-floating multipotent cell clusters that express the stem cell marker nestin during proliferation of neural stem cells. Neurospheres can be induced to differentiate into neuronal and glial phenotypes that express more mature cell type markers. This nestin⁺ mixed population of cells is referred to herein as primary NSCs. These primary NSCs were cultured on laminin-coated substrates for two days as described below, and immortalized so as to allow them to persist as nestin⁺, elongated cells for extended periods. The neurosphere clones that were obtained, including RG3.6, exhibited a bipolar morphology and were BLBP⁺, which are characteristics of radial glia.

The cells of the present invention may be derived from stem cells. In some embodiments, the cells are derived from neural stem cells (NSCs). In other embodiments, the cells are derived from embryonic stem cells. In still other embodiments, the cells are derived from adult stem cells. As used herein, the term “adult stem cells” is meant to include any adult stem cell sources such as bone marrow, astrocytes, cells from the SV zone, etc.

In one preferred embodiment, a radial glial cell population of this invention may be isolated from embryonic stem cells (ESCs). The ESCs can be induced to differentiate into a mixed population of cells including neural stem cells and glial phenotypes, referred to herein as primary NSCs. The primary NSCs can then be contacted with a protein that binds specifically to a marker on the cells of the present invention; and the bound cells can be isolated.

For example, as described above, GLAST is a marker characteristic of radial glia, and is expressed by the cells of the present invention. Therefore, an antibody that binds to a cell surface region of GLAST may be used to isolate the radial glial cells of this invention from primary NSCs. As described in further detail below, the present invention provides a monoclonal antibody that specifically recognizes an extracellular portion of GLAST, which may be useful in this regard. In one embodiment, this antibody specifically recognizes the region of GLAST corresponding to about amino acid 65 to about amino acid 82 of human GLAST (SEQ ID NO: 1).

### Methods of Use

The present invention provides methods of use of the cells of the present invention. Transplanted cells of this invention can be administered to any animal, including humans, with abnormal, neurological or neurodegenerative symptoms obtained in any manner.

As described in the Examples below, the present inventor has found that acutely following spinal cord injury, transplanted radial glia of this invention 1) form bridges that span across the injury site and expand into spared spinal cord tissue, 2) suppress deposition of chondroitin sulfate proteoglycans (CSPGs), which are factors associated with neural damage, 3) suppress macrophage infiltration, which may mediate inflammatory processes and secrete CSPGs, 4) promote preservation and organization of neurofilaments in the spinal cord following injury, and 5) promote functional recovery.

In one embodiment, the invention provides a method for protecting neural tissue in a human patient in need thereof. This method involves administering to the patient a therapeutically effective amount of an enriched population of the cells of the present invention.

The experimental studies presented herein show that the improved acute recovery after spinal cord contusion and transplantation of radial glia involves tissue protection. Since the radial glia were transplanted within thirty minutes following contusion and migrate extensively in white mater, they were well situated to protect white matter from secondary damage. As used herein, “white matter” is the portions of the spinal cord and brain which are white and composed of the long extensions (called axons) of neurons and their myelin sheaths.

Furthermore, behavioral scoring performed within the first week following injury showed significant improvement in rats transplanted with the inventive cells by comparison to rats that received fibroblasts or medium alone. Without being bound to any one theory, this early response was most likely due to acute morphological and biochemical changes produced by the inventive cells in and around the site of injury. The increases in neurofilament (NF) staining and organization observed in close association with the cells of this invention also provides evidence that radial glia are neuro-protective. This is likely to be a consequence of interactions between the inventive cells and neurons, consistent with their ability to promote cerebellar granule cell axonal growth in vitro (as described below).

Moreover, the cells of the present invention that survived following transplantation were located surrounding the injury site. In addition, many of these cells migrated into the white matter (myelinated regions) where they co-aligned with axons. Therefore, the inventive cells provide the physical support to protect neuronal axons.

Radial glia are known to express various transporters that can facilitate clearance of potentially excitotoxic chemicals. Therefore, transplants of cells of the present invention protect neurons from secondary damage. Interestingly, the radial glia of this invention suppressed deposition of CSPGs, including NG2, in and around contusion sites. These proteoglycans are known to inhibit axonal growth in vitro and in vivo. Thus, in one aspect of the present invention, the control exerted by the radial glia over the extracellular milieu facilitates survival and/or protection of neural tissue acutely. Such control directly or indirectly promotes axonal survival and axonal regrowth.

In a further embodiment, the invention provides a method for inhibiting macrophage infiltration in a patient with a spinal cord injury. Excessive infiltration of macrophages and other cells is likely to promote secondary damage and inhibit regeneration. Limiting the infiltration of cells into the injury site will help to facilitate regeneration and functional recovery following injury.

In a further embodiment, the invention provides a method for promoting axonal regrowth in a patient in need thereof. The axonal regrowth may include sprouting. Furthermore, the axonal regrowth may include regeneration. The method involves administering to the patient a therapeutically effective amount of an enriched population of radial glial cells according to the present invention.

In one embodiment, the administered cells enhance the organization of spinal cord tissue following injury. For example, the cells of the present invention can self-assemble to form scaffolds, or bridges that span across a spinal cord contusion site. These bridges facilitate migration of neurons and their processes. The experimental studies presented herein are the first to demonstrate that “bridging” of spinal cord lesions with radial glia is possible and promotes functional recovery.

In another embodiment, the administered cells suppress the deposition of CSPGs in the spinal cord tissue following the injury. CSPGs are believed to inhibit cell adhesion and axonal growth.

The cells of the present invention may be administered to a patient that has a neural injury. In some embodiments, the neural injury in the patient is a spinal cord injury or stroke.

The ability of the inventive cells to protect neural tissue and promote axonal growth in intact and injured spinal cords suggests that these cells are also useful for other CNS disorders. Therefore, in a further embodiment of this invention, the cells are administered to a patient that has a neurodegenerative disease. For example, the neurodegenerative disease may be selected from one of the following: Multiple Sclerosis, Amyotrophic Lateral Sclerosis, Parkinson's disease, Huntington's Disease and Alzheimer's Disease.

Cells can be delivered to any affected neural areas using any method of cell injection or transplantation known in the art.

The cells may, for example, be injected intrathecally into a neural injury site. Also, the cells may be injected intrathecally adjacent to a neural injury site. It is also possible to administer the cells intravenously or intraventricularly in order to treat a neural injury.

The cells may be injected intrathecally into or adjacent to a neurodegenerative disease site. Alternatively, the cells may be administered intravenously or intraventricularly in order to treat the neurodegenerative disease.

Cells injected to a particular neural region form a radial glial graft, which may have multiple functions. For example, in addition to the use of the cells for neural protection and/or axonal growth, it is also well within the contemplation of the present invention that the capacity of the inventive cells to generate oligodendrocytes and astrocytes is useful. For example, the cells of the present invention may be induced to differentiate into oligodendrocytes, allowing these cells to be used to repair demyelinating damage. Moreover, the cells may be induced to differentiate into astrocytes, allowing these cells to be used as a source of chemotactic and cell-surface signals to promote growth of axons.

The cells may be induced to proliferate and/or differentiate in vitro or in a human patient. In particular, it is contemplated that the cells can be induced to proliferate and differentiate in vitro, prior to administration to the patient. Moreover, the cells can proliferate in vitro prior to being administered, and to further proliferate and differentiate in vivo after being administered. Also, the cells can proliferate in vitro prior to being administered, and then differentiate in vivo without further proliferation after being administered. Alternatively, the cells can proliferate and differentiate in vivo after being administered.

The cells of the present invention may be transplanted as free cells. Alternatively, the cells may be contained within a cell encapsulation device, which may increase long-term in vivo cell survivability. Encapsulated cell therapy is discussed, for example, by Lanza and Chick in Sci. Amer: Sci. & Med., July/August, 16-25 (1995); and by Prakash and Soe-Lin in Trends Biomater. Artif. Organs, Vol. 18 (1), 24-35 (2004).

The cells of the invention can also be used to deliver therapeutic or other compounds, such as small molecules, peptides, proteins, etc. Examples of therapeutic agents include antimicrobial agents. In one embodiment, the antimicrobial agents are antibiotic or antiseptic agents, or combinations thereof. The antibiotic agents can be of the type including, but not limited to, ciprofloxacin, vancomycin, minocycline, rifampin and other like agents, as well as combinations thereof. Suitable antiseptic agents include, but are not limited to, the following: silver agents, chlorhexidine, triclosan, iodine, benzalkonium chloride and other like agents, as well as combinations thereof. Other examples of therapeutic agents are anti-thrombogenic agents, such as heparin, heparin derivatives, urokinase, and PPack (dextrophenylalanine proline, arginine, chloromethylketone); anti-inflammatory agents, such as dexamethasone, prednisolone, corticosterone, budesonide, estrogen, sulfasalazine, and mesalamine); anesthetic agents (such as lidocaine, bupivacaine, and ropivacaine); and anti-coagulants (such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin, prostaglandin inhibitors, platelet inhibitors and tick anti-platelet peptides).

Other therapeutic agents include growth factors (such as NGF, BDNF, NT3, NT4/5, ErbB2 (glia growth factor), PDGF and FGF. Further examples of therapeutic agents include cytokines (such as IL-6, LIF and CNTF). These factors have been found to promote nerve regeneration or the radial glial cell phenotype.

A therapeutic agent may be encapsulated in a cell encapsulation device along with the inventive cells. Alternatively, the cells may be genetically engineered to produce the therapeutic compound. For example, any gene encoding a therapeutic peptide or protein may be introduced into the cells. The genetically-engineered cells may be transplanted as free cells or may be contained within an encapsulation device.

Cells may be genetically transduced by any means known in the art, including calcium phosphate transfection, DEAE-dextran transfection, electroporation, lipofection, and the like. Any expression system known in the art can be used to express a therapeutic compound, provided it has a promoter that is active in the cells of the present invention, and appropriate internal signals for initiation, termination and polyadenylation.

Cells can be genetically modified to express growth factors including NGF, BDNF, NT3, NT4/5, ErbB (glial growth factor), PDGF and FGF, and/or cytokines including IL-6, LIF, CNTF that can be secreted from the cell. Bioactive fragments of various molecules including cell adhesion molecules, such as L1, can be expressed as fusion proteins with the constant region of immunoglobulins (Ig) know as Fc fusion proteins or receptor bodies. As described above, these factors have been found to promote nerve regeneration or the radial glial cell phenotype.

Suitable expression vectors for use in mammalian cells are known (e.g., see Ausubel, F. M. et al. (Eds.), Current Protocols in Molecular Biology. John Wiley & Sons, Inc., New York, 1999. Such vectors include well-known derivatives of SV-40, adenovirus, cytomegalovirus (CMV) retrovirus-derived DNA sequences.

Further eukaryotic expression vectors are known in the art (e.g., P. J. Southern and P. Berg, J. Mol. Appl. Genet. 1:327-341 (1982); S. Subramani et al, Mol. Cell. Biol. 1:854-864 (1981); R. J. Kaufmann and P. A. Sharp, “Amplification And Expression Of Sequences Cotransfected with A Modular Dihydrofolate Reductase Complementary DNA Gene,” J. Mol. Biol. 159:601-621 (1982); R. J. Kaufmann and P. A. Sharp, Mol. Cell. Biol. 159:601-664 (1982); S. I. Scahill et al, “Expression And Characterization Of The Product Of A Human Immune Interferon DNA Gene In Chinese Hamster Ovary Cells,” Proc. Natl. Acad. Sci. USA 80:4654-4659 (1983); G. Urlaub and L. A. Chasin, Proc. Natl. Acad. Sci. USA 77:4216-4220 (1980).

The expression vectors useful in the present invention contain at least one expression control sequence that is operatively linked to the DNA sequence or fragment to be expressed. The control sequence is inserted in the vector in order to control and to regulate the expression of the cloned DNA sequence. Examples of useful expression control sequences are the lac system, the trp system, the tac system, the trc system, the tet system, major operator and promoter regions of phage lambda, the control region of fd coat protein, the glycolytic promoters of yeast, e.g., the promoter for 3-phosphoglycerate kinase, the promoters of yeast acid phosphatase, e.g., Pho5, the promoters of the yeast alpha-mating factors, and promoters derived from polyoma, adenovirus, retrovirus, and simian virus, e.g., the early and late promoters or SV40, and other sequences known to control the expression of genes of prokaryotic or eukaryotic cells and their viruses or combinations thereof.

Once the gene is in such an expression vector, the gene product may be produced in a suitable expression host in either a constitutive or inducible manner. Useful expression hosts include mammalian cells, such as the transplanted cells of the present invention.

Transplanted cells can be identified by prior incorporation of tracer dyes, such as rhodamine or fluorescein-labeled microspheres, fast blue or bis-benzamide. Alternatively, transplanted cells can be identified by genetic markers incorporated by any genetic transduction procedure known in the art to allow expression of such enzymatic markers as β-galactosidase or alkaline phosphatase, or fluorescent markers such as GFP.

### Methods for Preparing the Inventive Cells

The present invention provides a method for enriching a population of the radial glial cells of this invention. In one preferred embodiment, the cells are isolated from a mixed population of cells. Selection may be achieved by use of a marker characteristic of the inventive cells. In some embodiments, the mixed population of cells is contacted with a molecule that binds specifically to the marker on the cells, and the bound cells are isolated from unbound cells. For example, cells expressing an antigen protein on their surface can be contacted with an antibody molecule. The antibody may be a labeled antibody. For example, in some embodiments, the antibody used to isolate the cells of this invention is fluorescently-labeled.

Procedures for isolation may include magnetic separation using antibody-coated magnetic beads, affinity chromatography, and “panning” with antibody attached to a solid matrix, such as a plate. Techniques providing accurate isolation include fluorescence activated cell sorters. Cells may be selected based on light-scatter properties, as well as their expression of various cell surface markers. These isolation procedures are well known in the art.

In one embodiment, an antibody specific for a marker expressed by the cells of this invention is directly or indirectly conjugated to a magnetic reagent, such as a superparamagnetic micro particle (microparticle). Direct conjugation to a magnetic particle can be achieved by use of various chemical linking groups, as known in the art. Alternatively, an antibody can be indirectly coupled to the magnetic particles. For example, an antibody can be directly conjugated to a hapten, and hapten-specific secondary antibodies are conjugated to the particles. Suitable haptens include digoxin, digoxigenin, FITC, avidin, biotin, etc. Methods for conjugation of the hapten to a protein are known in the art and kits for such conjugations are commercially available. The magnetic particles including the antibody conjugated thereto can then be combined with a cell sample. After a suitable period of time for complexes to form between the antibody and the antigenic marker (usually at least five minutes, and usually not more than one hour), the cells are subjected to immunomagnetic selection according to known methods. Other procedures for isolation are discussed below.

The cells of this invention are characterized by both the presence of markers associated with specific epitopic sites identified by antibodies and the absence of certain markers as identified by the lack of binding of certain antibodies. Therefore, selection of the inventive cells may be achieved by a combination of negative selection (removal of cells) and positive selection (isolation of cells). In some embodiments, cells which have bound specifically to a molecule, such as an antibody, are subsequently separated from the molecule. For example, as described above, magnetic beads coated with an antibody that specifically binds to a cell surface marker, characteristic of the inventive cells can be used to isolate them. Removal of the cells from the beads is possible by papain treatment.

In one desired embodiment, the selected cells are GLAST⁺ or are derived from GLAST⁺ cells. It is noted that GLAST⁺ cells may lose this marker after passaging. Cells of this invention that are GLAST⁺ can be contacted with a molecule, such as an antibody that binds specifically to GLAST. Cells that bind to the antibody are then isolated from unbound cells.

As stated above, the present invention provides a monoclonal antibody which specifically recognizes an extracellular portion of GLAST. This antibody is useful for isolating the radial glial cells of this invention.

In some embodiments, an antibody that recognizes a cell surface region of GLAST may be used to isolate the cells of this invention from a mixed population of cells. For example, the invention provides a method for isolating a GLAST⁺ radial glial cell population that includes providing a mixed population of GLAST⁺ and GLAST⁻ cells; binding the cells to an antibody that recognizes an extracellular portion of GLAST; and isolating the antibody-bound cells from unbound cells. Therefore, in one embodiment, the method includes isolating GLAST⁺ cells or an enriched population of GLAST⁺ cells. The bound cells may then be separated from the antibody, if desired.

In one embodiment, the cells of this invention are isolated from a population of neural stem cells. In another embodiment, the cells are isolated from a population of adult neural stem cells, including any adult neural stem cell sources, as described above. In still another embodiment, the cells are isolated from a population of embryonic stem cells following differentiation into a population of cells containing radial glial neural stem cells.

The cells may be separated from a mixed population of stem cells by a fluorescence activated cell sorter (FACS) or other methodology having high specificity. Multi-color analysis may be employed, if desired. For example, in a first separation, which preferably starts with about 1-10×10⁶ cells, an antibody for nestin or BLBP, which are characteristic of the inventive cells, may be labeled with one fluorochrome, while antibodies for other lineages may be conjugated to a different fluorochrome. Fluorochromes which may find use in multi-color analysis include phycobiliproteins, e.g., phycoerythrin and allophycocyanins, fluorescein, Texas red, etc. While each lineage may be separated in a separate step, desirably the lineages are separated at the same time as one is positively selecting for Glast. BLBP or nestin can then be used as intracellular markers to verify the FACS isolation procedures using a fraction of the cells for analysis.

The cells may be selected against dead cells by employing dyes associated with dead cells (propidium iodide, LDS). Other techniques for positive selection may be employed which permit accurate separation, such as affinity columns, and the like.

As described above, the inventive cells may be isolated by binding them to antibodies specific for markers and isolating the bound cells from a mixed population of cells. Alternatively, a mixed population of cells can include a reporter indicating transcription of the genetic promoter region for a marker associated with the cells of this invention. In one embodiment, the marker is a protein, such as BLBP, GLAST or nestin, which are characteristic of the inventive cells. The mixed population of cells can be transfected with a DNA molecule that includes a reporter gene (e.g. GFP) operatively linked to the promoter for a marker associated with the inventive cells, such as BLBP, GLAST or nestin.

### Antibodies

This invention provides monoclonal antibodies that bind specifically to an antigenic determinant characteristic of the cells of the present invention. In particular, the invention provides a monoclonal antibody that specifically recognizes an extracellular portion of GLAST. In one embodiment, the monoclonal antibody is raised against human GLAST. Typically, approximately six amino acids or greater form an antigenic determinant. In one embodiment, the monoclonal antibody of this invention specifically recognizes the region of GLAST corresponding to about amino acid 65 to about amino acid 82 of human GLAST (SEQ ID NO: 1).

In this specification, an antibody is defined broadly as a protein that binds specifically to an epitope. Antibodies that bind specifically to an epitope may comprise an antibody hypervariable region. The hypervariable region may further comprise an entire antibody variable region. The antibody variable region may further comprise an antibody constant region. The molecule that comprises an antibody hypervariable region may be an antibody including a whole antibody, an antibody fragment, a chimerized antibody or a humanized antibody. The antibody of the present invention is monoclonal.

Suitable variable and hypervariable regions of non-human antibodies may be derived from antibodies produced by any non-human mammal in which monoclonal antibodies are made. Suitable examples of mammals other than humans include, for example, rabbits, rats, mice, horses, goats, or primates. Preferably, the antibodies are human antibodies. The antibodies may be produced in a transgenic mouse. An example of such a mouse is the so-called XenoMouse™ (Abgenix, Freemont, Calif.) described by Green, L L., “Antibody Engineering Via Genetic Engineering of the Mouse: XenoMouse Stains are a Vehicle for the Facile Generation of Therapeutic Human Monoclonal Antibodies,” J. Immunol. Methods,” 10;231(1-2): 11-23(1999).

Antibodies fragments have binding characteristics that are the same as, or are comparable to, those of the whole antibody. Suitable fragments of the antibody include any fragment that comprises a sufficient portion of the hypervariable (i.e. complementary determining) region to bind specifically, and with sufficient affinity, to GLAST.

The fragments may be single chain antibodies. Single chain antibodies are polypeptides that comprise at least the variable region of the heavy chain of the antibody and the variable region of the light chain, with or without an interconnecting linker.

A chimerized antibody comprises the variable region of a non-human antibody and the constant region of a human antibody. A humanized antibody comprises the hypervariable region (CDRs) of a non-human antibody. The variable region other than the hypervariable region, e.g. the framework variable region, and the constant region of a humanized antibody are those of a human antibody.

The antibodies and functional equivalents may be members of any class of immunoglobins, such as: IgG, IgM, IgA, IgD or IgE, and the subclass thereof. The functional equivalents may also be equivalents of combinations of any of the above classes and subclasses.

Monoclonal antibodies may be produced by methods known in the art. These methods include the immunological method described by Kohler and Millstein in Nature 256, pp 495-497 (1975) and by Campbell in “Monoclonal Antibody Technology”, The Production And Characterization Of Rodent And Human Hybridomas” in Burdon, et al. (Eds.), Laboratory Techniques in Biochemistry and Molecular Biology, 13, Elsevier Science Publishers, Amsterdam (1985); and Coligan, J. E., et al. (Eds.), Current Protocols In Immunology, Wiley Intersciences, NY, (1999); as well as recombinant DNA methods described by Huse, et al., Science, 246, pp 1275-1281 (1989). The recombinant DNA method preferably comprises screening phage libraries for human antibody fragments.

In order to produce monoclonal antibodies, a host mammal is inoculated with a GLAST peptide or peptide fragment, and then boosted. Spleens are collected from inoculated mammals a few days after the final boost. Cell suspensions from the spleens are fused with a tumor cell in accordance with the general method described by Kohler and Millstein in Nature, 256, pp 495-497 (1975).

If the fragment is too short to be immunogenic, it may be conjugated to a carrier molecular. Some suitable carrier molecules include key hold limpet, hemocyanin and bovine serum albumin. Conjugation may be carried out by methods known in the art (Coligan, J. E., et al. (Eds.), Current Protocols In Immunology, Chapter 9, Wiley Intersciences, NY, (1999). One such method is to combine a cysteine residue of the fragment with a cysteine residue on the carrier molecule.

### EXAMPLES

### Example 1

### Preparation and Maintenance of Radial Glial-Like Cell Clones

This example describes the procedure used to generate clones from embryonic rat cortex that exhibited properties of radial glia.^{19,21} The clones were generated by immortalizing them with v-myc retrovirus²⁰.

Forebrains of E14.5 rat embryos were dissected and the meningeal membranes were removed. Tissues were dissociated into single cell suspensions using fire-polished Pasteur pipettes and 10⁶ cells were incubated on 10 cm dishes in DMEM/F12 (Invitrogen) supplemented with 25 mM glucose (Sigma), 2 mM glutamine (Invitrogen), penicillin/streptomycin (Invitrogen), 10 ng/ml FGF2 (BD Biosciences), 2 μg/ml heparin (Sigma) and 1×B27 (Invitrogen). Cells that propagated as neurospheres for 2 days were dissociated again by mild trypsinization (0.025% for 5 min). This yielded a population of cells that was nearly all nestin⁺, consisting of a combination of NSC and progenitors that will be referred to here as primary NSCs.

For immortalization, the primary NSCs were cultured on laminin-coated substrates for two days, and infected with PK-VM-2 retrovirus as described^{20,21}. After 4-5 days in selection in 200 μg/ml G418 (Invitrogen), individual cells were cloned at limiting dilution in 96 well plates. 36 neurosphere clones were obtained and upon passage nine clones were selected that contained only BLBP+ cells that were negative for NeuN and GFAP. Of these, four clones called RG1.9, RG3.6, RG3.7 and RG4.7 with a polarized morphology were selected for further analysis.

For differentiation in vitro, at passage (P) 7, RG3.6 cells and P7 NSCs were each cultured on laminin-coated coverslips in FGF2 (10 ng/ml, BD Biosciences) containing serum-free DMEM/F12 (Invitrogen) medium for 1 day, then the medium was replaced with culture medium lacking FGF2, including 1% fetal bovine serum (FBS), for 6 days, and the cultures were fixed, and immunostained.

To maintain the RG3.6 cells in culture (self-renewing conditions), RG3.6 cells are cultured as neurosphere in FGF2 (10 ng/ml, BD Biosciences) containing serum-free DMEM/F12 (Invitrogen) medium and passaged following every 3-5 by dissociation with mild trypsinization (0.025% for 5 min). Fresh FGF2 is added daily to a final concentration of 10 ng/ml.

### Example 2

### Procedure for Immunostaining Radial Glial Cells of the Present Invention

Among the four representative clones isolated in Example 1, clone RG3.6 was selected for detailed immunostaining, since it showed desirable behavior following transplantation into the adult spinal cord, as described below. The present example describes the immunostaining and analysis used to characterize these cells with respect to cell type specific markers.

### Immunofluorescence

RG3.6 neurospheres were dissociated and plated on coverslips coated with 20 μg/ml laminin. Cells were fixed with 4% paraformaldehyde for 15 minutes at room temperature and washed with PBS. Cells were incubated with one of the following primary antibodies in 10% normal goat serum and 0.3% TritonX100 in PBS for 2 hours at room temperature: mouse monoclonal anti-nestin at 1:40 (Developmental Study Hybridoma Bank, Iowa City, Iowa), rabbit polyclonal anti-BLBP at 1:1000, rabbit polyclonal anti-GFAP at 1:200 (R401), mouse monoclonal anti-TuJ1 at 1:200 (Chemicon, Temecula, Calif.), and mouse monoclonal anti-GalC at 1:50. After washing, cells were incubated with appropriate secondary antibodies at 1:200 (Molecular Probes, Eugene, Oreg.) for 1 hour at room temperature: Alexa 568 conjugated goat anti-mouse IgG and Cy5 conjugated goat anti-rabbit IgG (Jackson Immunoresearch, West Grove, Pa.). Cells were washed, nuclei were labeled with Hoechst 33342 at 1:2000 (Sigma-Aldrich, St. Louis, Mo.) and coverslips were mounted with Gel/Mount (Biomedia, Foster City, Calif.)

### Quantitation of Cells Differentiated In Vitro

Images acquired on Zeiss Axiophot microscope were analyzed in Adobe Photoshop program. The total number of cells was determined by counting the total number of nuclei that stained with Hoechst dye. A cell was identified as positive for a cell type specific marker TuJ1, Nestin or GFAP) if the red signal stained both the processes and the outline of the nucleus of that particular cell. Percentage of positive cells was calculated and presented at 1 and 2 weeks after differentiation.

### Example 3

### Procedure for Transplanting Radial-Glial Cells of the Present Invention Into Intact Spinal Cord and Cerebellum

Intact spinal cord: A total of 18 adult female Sprague-Dawley (SD) rats (Taconic, Germantown, N.Y.) weighing 200-250 g were used in this study. Animals were anesthetized with intraperitoneal injection of pentobarbital (35 mg/kg) and laminectomies were performed to expose thoracic segments T9-10. RG3.6 or NSC cells were dissociated with trypsin into single cells in DMEM+F12 medium and resuspended at a density of 1×10⁵ cells/ml. Cells were injected slowly during a time course of 10 minutes into ventral column of T10 using a sterile glass tip with diameter 50 mm connected to a 5 μl Hamilton syringe. After injections, the muscle and skin were sutured separately. Cefazolin (25 mg/kg) was administered for 7 days after surgery to prevent infection. Cyclosporin A (Sandoz Pharmaceuticals, East Hanover, N.J.) was administered subcutaneously at a dose of 1 mg/100 g body weight throughout the survival period and animals were sacrificed at different time points (1 wk, n=6; 2 wk, n=6; and 4 wk, n=6).

Cerebellum: P3 rat pups were rendered unconsciousness by chilling the animals at 4° C. for 1-2 minutes, a small incision was made in the skin and a Hamilton syringe needle was lowered gently through the incision to a position just beneath the meninges. 2.5×10⁴ RG3.6 cells were injected slowly on each side of the cerebellum. After withdrawing the needle, the skin incision was closed with 7-0 silk suture (Ethicon). The animal was warmed to 35.5° C. and returned to the litter. They were sacrificed after 3 days.

### Example 4

### Procedure for Assessing Differentiation of Radial Glial Cells of the Present Invention In Vivo After Implantation into Intact Spinal Cord

To study differentiation in vivo, GFP-labeled cells were counted that were positive for nestin (a radial glial cell marker) or positive for GFAP (an astrocyte cell marker) at four weeks after implantation of RG3.6 or GPF-NSC into intact spinal cord. Tissue processing and immunostaining were described previously¹⁷. An average of 300 GFP-labeled cells were counted for each animal. Expression levels of Hoescht+ GFP-labeled cells were scored as: ++, strong positive cells; +, weak positive cells; −, negative cells; or unscored cells.

### Example 5

### Procedures for Contusion Injury Followed by Transplantation of Radial Cells of the Present Invention

A total of 46 adult female SD rats weighing 200-250 g were used in this study including 16 to obtain preliminary observations to optimize cell dosing using 0.5-2×10⁵/μl. Animals were anesthetized and the spinal cord was exposed by laminectomy on T9-10. Contused spinal cords were produced by dropping a 10.0-g rod onto the exposed spinal cord from a height of 12.5 mm using the MASCIS impactor²². After cell injections (Experiment 1 or 2 below), muscles and skin were closed separately. Cyclosporin A (Sandoz Pharmaceuticals, East Hanover, N.J.) was administered subcutaneously at a dose of 1 mg/100 g body weight throughout the survival period. Animals were sacrificed 6 weeks after the contusion injury and spinal cords were treated as described²³.
Experiment 1. A total of 4 μl (2 μl at the center of the contusion site and 1 μl at 2 mm rostral and caudal sites from the center) of RG3.6 cells (1×10⁵/μl) or GFP rat skin fibroblasts (0.5×10⁵/μl) were injected (n=10 for RG3.6, n=10 for GFP fibroblasts).
Experiment 2. Cell injection procedures were the same as described in Experiment 1. In Experiment 2, 6 animals received RG3.6 transplants and 4 received vehicle medium (DMEM+F12) after contusion injury.

After the animals were sacrificed, half of the spinal cords were cut sagittally at 20 μm as cryostat sections and the other half were cut horizontally at 7 μm as paraffin sections. To quantify the number of neurofilament-stained axons at the epicenter of contusion site, axons were counted in every sixth section for cryostat sections and in every fifteenth section for paraffin sections. Experiment 1 and 2 were performed by different surgeons. Locomotor recovery for experiments 1 and 2 was assessed weekly, using the 21-point BBB score^{24,25} by two separate BBB scoring teams that were unaware of experimental treatments.

### Example 6

### Procedures for Immunostaining Spinal Cord Tissue to Assess Transplantation of Cells of the Present Invention into Intact and Injured Spinal Cords

### Histological Procedures

Animals were euthanized with an intraperitoneal injection of sodium pentobarbital (50 mg/kg), followed by transcardial perfusion with 4% paraformaldehyde in 0.1 M PB after vascular washout with PBS. The spinal cords were removed, post-fixed overnight in the same fixative, cryoprotected with 20% sucrose overnight, and embedded in OCT compound (Fisher Scientific, Pittsburgh, Pa.). Horizontal (for transplantation in intact spinal cord) or sagittal (for transplantation in injured spinal cord) sections were cut at 20 μm with a cryostat (Hacker) and mounted on Superfrost Plus Microscope Slides (Fisher brand). Cerebella were fixed similarly except they were embedded in 3% agarose and 100 mm sections were cut with a Vibratome. For immunostaining, slides were blocked with 10% normal goat serum (NGS), 0.3% Triton X-100 in PBS for 2 hours at room temperature and incubated overnight at 4° C. with one of the following primary antibodies: rabbit polyclonal anti-GFAP at 1:200, mouse monoclonal anti-nestin at 1:40, mouse monoclonal anti-CS56 at 1:400 ( Sigma-Aldrich, St. Louis, Mo.), rabbit polyclonal anti-NG2 at 1:500, mouse monoclonal anti-rat ED1 at 1:300 (Serotec, Raleigh, N.C.) and mouse monoclonal anti-neurofilament at 1:500 (Clone N52, Sigma-Aldrich, St. Louis, Mo.). Sections were washed with PBS and incubated with appropriate secondary antibodies at 1:400 dilution for 1 hour at room temperature: Alexa 568 conjugated goat anti-rabbit IgG, Alexa 568 conjugated goat anti-mouse IgG, Alexa 488 conjugated goat anti-mouse IgG, Cy5 conjugated goat anti-rabbit IgG (Jackson Immunoresearch, West Grove, Pa.). Sections were washed with PBS and counterstained with Hoechst 33342 (Sigma-Aldrich, St. Louis, Mo.) and mounted with Gel/Mount or ProLong antifade mounting medium (Molecular Probes). Analysis of the images was performed using Zeiss LSM 510 confocal laser scanning microscope (LSM). Intact spinal cords were imaged with a Cool Snap Pro camera (Media Cybernetics) using a Zeiss Stemi II microscope equipped with fluorescence optics.

### Quantification of Immunostaining

Quantification of spinal cord area immunolabeled for CS56, NG2 and NF was performed using Zeiss 510 LSM confocal software. Two Para-sagittal sections close to the midline were used for each animal. Tiled images composed of 30 10× images with a frame size of 2.7 mm×9.2 mm was employed to cover ˜a 9 mm length in sagittal sections. All staining was performed under the same conditions and images were acquired with invariant parameters using the Zeiss 510 system. Staining intensity thresholds for each antibody were determined after all images were acquired to optimize the signal noise ratio or each antibody. Over the range of intensities from 0-255, thresholds were: 50 for CS56, 60 for NG2, and 80 for NF. Areas with intensities higher than thresholds were recorded and normalized to the total areas measured to obtain the percent of labeling. Regions of meninges and dorsal root ganglion were excluded. Quantification of NF staining in cryosections was performed similarly except the tiled images consisted of 6 images covering the injury center. For paraffin sections, the numbers of filaments in mid-sagittal sections was counted along the dorsal-ventral axis at the center of the injury site. 1 mm lengths of dorsal and ventral white matter were outlined, excluding meninges, dorsal root ganglia, cysts and central fibroblast masses where cysts typically form.
Statistical analysis: Data in each analysis are expressed as the mean±SEM. Differences among the means were evaluated by Fisher's Protected Least Significant Difference(PLSD). *, p<0.05; **, p<0.01; ***, p<0.001.

### Example 7

### Characterization of Radial Glial Cells of the Present Invention

Molecular Characteristics of Undifferentiated vs. Differentiated Cells

In order to study radial glia in vivo, a set of clones was derived from E13.5 GFP rat cortical neurospheres, as described in Example 1. These clones exhibited properties of radial glia¹⁹. After selection with G418, individual clones were cloned and most including RG3.6 had a bipolar morphology. Among the clones that were generated, many were found to express the radial glial markers BLBP, nestin and vimentin. Four representative clones were tested for their behavior following transplantation into adult spinal cord and all migrated robustly along the rostral-caudal axis in white matter tracts. Among these clones, clone RG3.6 was selected for detailed examination, since it showed extensive migration and no tendency for mass formation in spinal cord.

Based on immunofluorescence analysis (Example 2), the group of clones exemplified by RG3.6 cells were found to express markers for radial glia, but not for neurons (NenN and β-III tubulin), oligodendrocytes (O4) and astrocytes (GFAP). These results are summarized in Table 1 below.

TABLE 1
Molecular Characteristics of Undifferentiated Radial Glial Cells
of the Present Invention

| | |
|---|---|
| Nestin | + |
| BLBP | + |
| Vimentin | + |
| NenN | − |
| β-III tubulin (TuJ1) | − |
| O4-Antigen | − |
| GFAP | − |

Considering evidence that radial glia are NSC that can give rise to neurons and glia^{10,11}, the present inventors examined the differentiation of RG3.6 cells in vitro, as described in Example 1. Upon withdrawal of FGF2 from the culture medium, these cells differentiated within 4 days into neurons, astrocytes and oligodendrocytes, with astrocytes being the most prevalent differentiated cells obtained. The addition of FBS during differentiation increased the proportion of astrocytes and decreased the proportion of oligodendrocytes, while more oligodendrocytes were obtained without serum or FGF2, and more neurons with retinoic acid (data not shown)^{26,27}. The molecular characteristics after differentiation of the inventive cells in vitro are summarized below in Table 2.

TABLE 2
Molecular Characteristics After Differentiation of Radial Glial Cells
of the Present Invention In Vitro

| | |
|---|---|
| β-III tubulin (TuJ1) | + |
| GFAP | + |
| Galactocerebroside (GalC) | + |

RG3.6 also shares these properties with clone L2.3¹⁹, as well as the ability to support neuronal migration in vitro (data not shown). Following transplantation into early postnatal cerebellum, RG3.6 cells coaligned with nestin+ radial glia. Thus, RG3.6 cells exhibited morphological, molecular and developmental properties of radial glia¹².

Comparison of the Differentiation In Vitro of Cells of the Present Invention with Rat Cortical NSCS

One rationale for introducing v-myc into NSCs was to inhibit their ability to differentiate²⁰. To determine whether this was true, the present inventor compared the differentiation of RG3.6 cells with noninfected NSCs. These results are shown in Table 3 below.

TABLE 3
Comparison of Differentiation of Radial Glial Cells of the
Present Invention With NSC in Vitro

| | Week 1 | | Week 2 | |
|---|---|---|---|---|
| | NSC | RG3.6 | NSC | RG3.6 |
| Nestin | 60.4% | 97.0% | 41.6% (n = 505) | 44.8% |
| | (n = 197) | (n = 98) | | (n = 2019) |
| TuJ1 | 23.5% | 0.5% (n = 227) | 21.0% (n = 536) | 2.0% |
| | (n = 174) | | | (n = 1837) |
| mGFAP | 4.7% | 0.2% (n = 443) | 50.0% (n = 580) | 20.6% |
| | (n = 137) | | | (n = 2004) |

As shown in Table 3, within 2 weeks in the absence of FGF2, most NSCs differentiated into astrocytes expressing GFAP and fewer cells differentiated into neurons expressing TuJ1. In addition, there was a decrease in the intensity of nestin staining, as well as in the numbers of nestin+ cells obtained from the primary NSCs. In contrast, the RG3.6 cells showed much less or slower differentiation into GFAP+ astroglia and TuJ1+ neurons following incubation in identical differentiation conditions for 1 and 2 weeks. In addition, loss of nestin was much slower in the RG3.6 cells particularly at 1 week and the nestin+ RG3.6 cells at 2 weeks were typically bipolar like radial glia in contrast to the more astrocytic morphology observed for the NSCs. Another v-myc transduced clone called RG4.7 behaved similarly to RG3.6 upon differentiation (data not shown). These results indicate that cells of the present invention differentiate slower in vitro than rat cortical NSCs

Comparison of the Differentiation In Vivo of Cells of the Present Invention with Rat Corticol NSCs

The differentiation of these cells in the adult spinal cord was compared to evaluate their potential use for transplantation following spinal cord injury using the procedure described in Example 4 above. One week following transplantation into normal adult spinal cord, RG3.6 and NSC migrated robustly along the rostral-caudal axis in the white matter and much less migration was observed into grey matter¹⁷. Both the RG3.6 cells and the GFP-NSCs were nestin+ and exhibited bipolar morphologies with cells in white matter aligned along the rostral-caudal axis. By four weeks following transplantation, most of the GFP-NSCs did not stain for nestin in contrast to the RG3.6 cells, most of which still expressed nestin. Moreover, at 4 weeks the RG3.6 cells retained their bipolar morphology while the GFP-NSCs exhibited complex morphologies characteristic of more differentiated cells. Many of these complex cells exhibited multiple processes that were GFAP+, suggesting that many GFP-NSCs differentiated into astrocytes.

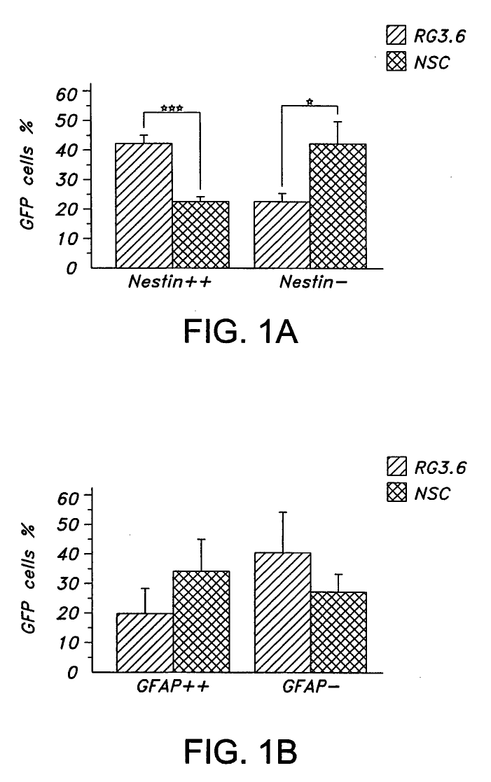

To compare the differentiation of NSC with RG3.6 in vivo, the percent of Hoechst+/GFP+ cells that expressed nestin (a marker for radial glia) or GFAP (a marker for astrocytes) was analyzed. These results are shown in FIG. 1. The percent of GFP+ cells that did not differentiate was quantified as the fraction that were nestin+. Twice as many strongly nestin+ cells were found with RG3.6 than with NSCs. In a complementary manner, the percent of nestin− cells was higher for the NSCs than with RG3.6. This analysis yielded statistically significant differences and was fairly straightforward given that there was little endogenous nestin staining in the adult spinal cord. The percent of GFAP+ cells was also quantified as a measure of cell differentiation since most cells that differentiated did so along the astrocytic lineage. However, this was much more complex given the widespread expression of GFAP by endogenous astrocytes in the spinal cord. Nevertheless, similar patterns of differentiation were found insofar as a higher percentage of NSCs was strongly GFAP+ and a higher percentage RG3.6 cells was GFAP− (FIG. 1).

In summary, the results of FIG. 1 show that more RG3.6 cells are nestin⁺ and more NSC are GFAP⁺. This indicates that, as compared to NSCs, cells of the present invention are inhibited from differentiating into glial cells, such as astrocytes in a normal spinal cord.

### Transplanted Cells Bridge Spinal Cord Lesions and Promote Functional Recovery

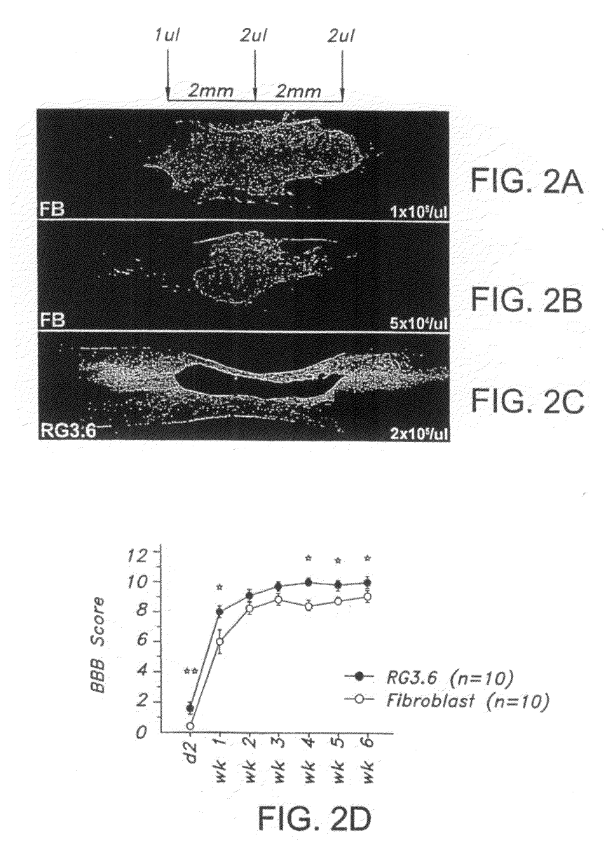

Contusion Experiment 1: RG3.6 cells vs. fibroblasts. GFP-fibroblasts (control) or RG3.6 cells (one embodiment of the radial glial cell population of the invention) were implanted at 3 positions (at the center of the injury site and ˜2 mm rostral and caudal) immediately following contusion of adult rat spinal cords with the MASCIS Impactor. Histological analysis (performed as described in Example 6) showed that robust GFP fluorescence persisted for both types of cells but the fibroblasts remained almost exclusively in the spinal cord injury site (FIG. 2A,B). Remarkably, the RG3.6 cells were observed several mm from the injection site, indicating they had migrated rostrally and caudally usually into white matter tracts where they were aligned along the rostral-caudal axis (FIG. 2C). By 3 weeks following contusive injury to the rat spinal cord, a cavity typically forms, but these cysts were not observed with the fibroblast implants as the cells appeared to fill the injury site (FIG. 2A,B). Cysts also formed with the RG3.6 cell implants but they were surrounded by the RG3.6 cells that in most cases formed a continuum across the injury site at 6 weeks (FIG. 2C).

Preliminary experiments were performed to optimize cell dosing. 6 weeks after transplant using 1-2×10⁵ cells/μl, fibroblasts filled the cavity and often formed bulges in the spinal cord that were not observed with RG3.6 cells. Reducing fibroblasts to 0.5×10⁵ cells/μl eliminated the bulging. Besides differences in cell migration in the spinal cord, the larger size of the fibroblasts may contribute to the distention of the spinal cord with fibroblasts that was not observed with similar or double doses of radial glia. Because of potential complications of the mass effect with the fibroblast transplants, half as many fibroblasts were used in the behavioral studies.

BBB behavioral scoring was performed weekly and the rats were sacrificed after 6 weeks. Functional analysis indicated that BBB scores were consistently higher for the rats that received RG3.6 cells than for those that received fibroblasts (FIG. 2D). Interestingly, significant differences in BBB scores were observed very early following contusion (e.g. at 2 and 7 days)—too early to result from regeneration, suggesting that the RG3.6 cells protected the spinal cord in some manner.

A common feature of cell transplants that was observed with both fibroblasts and RG3.6 cells was a variably reduced degree of cord shrinkage at the injury site. Although implantation of various types of cells could improve recovery by simply replacing lost cells and preventing cord shrinkage, the behavioral scoring results indicated a statistically significant improvement of the RG3.6 cells vs. the fibroblasts (FIG. 2D), suggesting that the RG3.6 cells had particular properties that were beneficial.

Because the fibroblasts filled the lesion site, it was also of interest to analyze the implantation of RG3.6 cells vs. a non-cell control treatment, i.e. injection of medium alone. This experiment also allowed the present inventor to address potential beneficial or detrimental affects of non-neural cell transplants into the spinal cord.

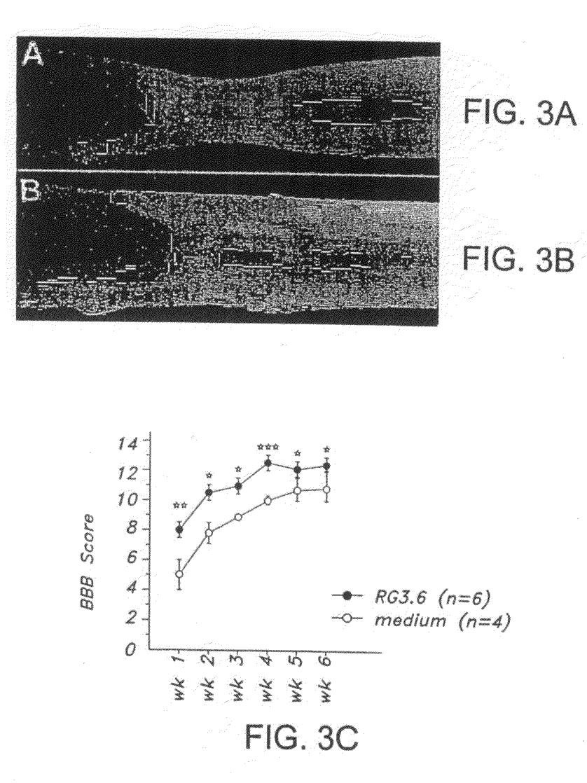

Contusion Experiment 2: RG3.6 vs. medium control. Immediately following contusion of adult rat spinal cords, RG3.6 cells or medium was injected at 3 positions (center of injury and ˜2 mm rostral and caudal). Gross anatomical analysis after 6 weeks showed shrinkage of the spinal cord injected with medium only, while the RG3.6 injected cords exhibited much less or no detectable shrinkage (FIG. 3A,B). Fluorescence imaging (not shown) of the intact cords showed the presence of RG3.6 cells (identified by their GFP fluorescence) that could often be seen surrounding the injury site. A lower GFP intensity that is likely to represent the cystic cavity was observed at the center of injury.

Functional analysis indicated that BBB scores were consistently higher for the rats that received RG3.6 cells than for those that received only medium (FIG. 3C). As in contusion Experiment 1, differences in BBB scores were observed very early following contusion, consistent with a protective effect on the spinal cord following contusive injury.

### Accumulation of Chondroitin Sulfate Proteoglycans Following Spinal Cord Injury is Inhibited by Cells of the Present Invention

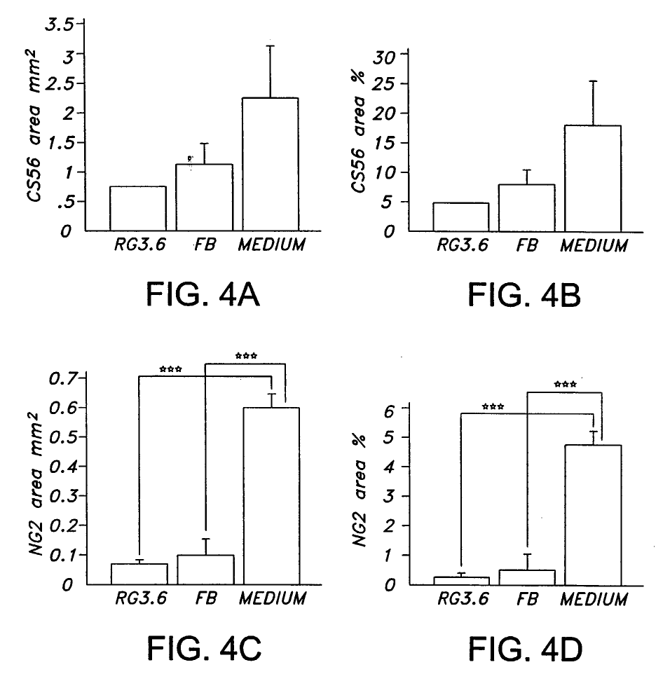

The early improvement in behavioral scores with RG3.6 cell transplants (FIGS. 2 and 3) suggested that the spinal cord was being protected from secondary damage. Associated with neural tissue damage, one typically finds various factors including accumulations of chondroitin sulfate proteoglycans (CSPG), which are believed to inhibit cell adhesion and axonal growth²⁸⁻³¹. Immunostaining confirmed extensive deposits of CSPGs in the spinal cord following contusive injury. In contrast, there was much less CSPG deposition following transplantation of RG3.6 cells or fibroblasts. These results are shown in FIG. 4(A and B). Among these CSPGs, NG2 expression is best correlated with the injury site with contributions from several local sources including reactive astrocytes, oligodendrocyte precursors, and macrophages³⁰. The enhanced accumulation of NG2 was confirmed in and around the injury site, with expression decreasing to negligible levels in rats that received RG3.6 cell transplants, as shown in FIG. 4(C and D).

Interestingly, rats that received transplants of fibroblasts also showed low levels of CSPG deposition, but their BBB scores were inferior to rats transplanted with RG3.6 cells. Thus, inhibition of CSPG deposition alone can not explain the functional recovery achieved with RG3.6 cells, since similar reductions were achieved in fibroblasts implants.

### Cells of the Present Invention Inhibit Macrophage Infiltration in Spinal Cord Following Injury

Given that macrophages are a major source of CSPGs in the injured spinal cord, the inhibition of CSPG deposition in rats injected with RG3.6 cells raised the possibility that RG3.6 cells may interfere with macrophages. Immunostaining for activated macrophages with monoclonal antibody ED1 revealed very few reactive macrophages in rats treated with RG3.6 or fibroblasts in contrast to the large numbers detected in contused rats treated only with medium. The few macrophages that were detected in mid-sagittal sections in RG3.6-injected or fibroblast-injected rats were in complementary patterns with the implanted cells. In more lateral sections where there were fewer RG3.6 cells, there were greater numbers of macrophages than near the midline in non-overlapping patterns.

The results suggest that implantation of RG3.6 cells can inhibit infiltration of macrophages and this likely explains the dramatic inhibition in CSPG levels. The mutually exclusive patterns also suggest that where macrophages do infiltrate, they can exclude other cells perhaps as a consequence of local deposition of CSPGs. The exclusion of macrophages likely helps to preserve axons and promote axonal growth or regeneration.

### Cells of the Present Invention Preserve Neurofilaments in Spinal Cord Following Injury

Behavioral outcome was improved in spinal cord contused rats—even at early times following transplantation of RG3.6 radial glial cells by comparison to fibroblasts or medium (see FIGS. 2 and 3). Histological analyses indicated that cyst formation did not correlate with recovery, as cysts did not form when fibroblasts were implanted, but did develop in RG3.6 treated rats. Although CSPG deposition did not correlate strictly with functional recovery as noted above, it is likely to be a contributing factor that may account for some improvement in recovery with fibroblasts. To determine whether radial glia promoted axon sparing or regeneration, immunostaining for neurofilament proteins (NF) was performed. By comparison to implants of fibroblasts or medium, spinal cords that received RG3.6 cells showed improved tissue architecture and NF alignment. Rats treated with RG3.6 cells exhibited longitudinally aligned bundles of NF most prominently in regions where the RG3.6 cells had infiltrated, in contrast to rats treated with fibroblasts or medium alone, where NF were disorganized. There was little or no NF staining in cysts that formed in rats treated with RG3.6 or medium. In contrast, most fibroblast-treated rats did not form cysts, but had disorganized NF in the injury site.

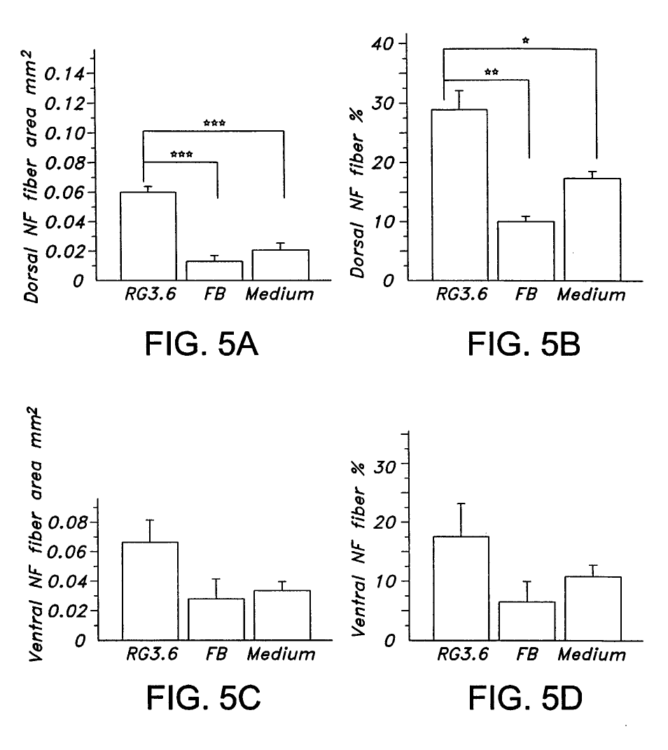

RG3.6 cells identified by their intrinsic GFP fluorescence revealed that the implanted cells surrounded the injury site (see FIG. 2C) and typically migrated into adjacent white matter tracts including the ventral and dorsal fiber tracts. These regions often contained longitudinally elongated GFP+ cells, suggesting the local infiltration and migration of the RG3.6 cells. Remarkably, the NF staining in these regions was fairly dense with the majority of filaments coaligned with the RG3.6 cells. Quantitative measurements of the numbers of NF+ filaments observed in mid-sagittal sections indicated on average 42% more filaments in cords implanted with RG3.6 cells (524+30) than cords implanted with medium (369+22). Areas occupied by NF+ cells in dorsal fiber tracts were statistically greater for RG3.6 treated rats than rats treated with fibroblasts or medium alone (FIG. 5A and B). A similar trend was observed in ventral fiber tracts (FIG. 5C and D). The results indicate that NF organization and number was increased following RG3.6 transplant into contused spinal cords. Therefore, RG3.6 cells promote white matter sparing.

The close co-alignment of NF+ fibers with GFP+ radial glia and the increase in the area of NF+ staining suggests that RG3.6 radial glia can interact with axons and may promote axonal growth. To test this hypothesis, granule cell neurons were cultured on monolayers of RG3.6 cells and fibroblasts. Granule neurons extended long processes on monolayers of RG3.6 cells, but they did not on fibroblasts where they tended to associate with each other. These results suggest that RG3.6 cells provide a favorable substrate for neuronal interaction that can support axonal outgrowth.

### Example 8

### Procedure for Fluorescence Activated Cell Sorting (FACS) of ESC-Derived NSC Cells

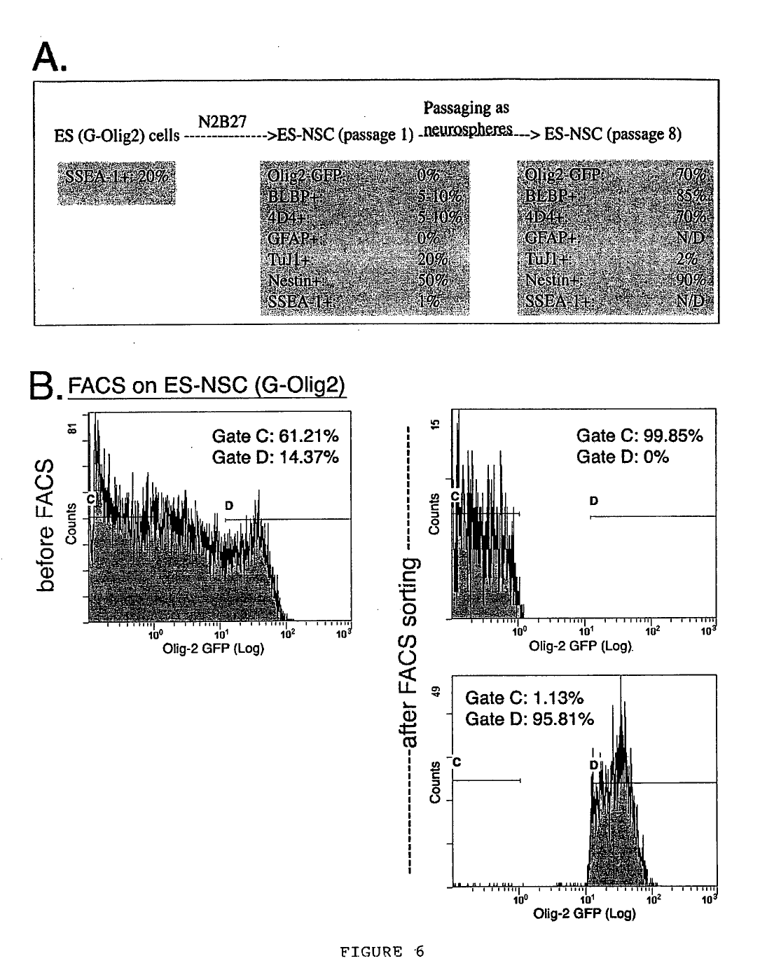

This example describes the generation of NSC from ES cells (FIG. 6A) and isolation of NSC by FACS (FIG. 6B).

G-Olig2 ESC (Xian et al., 2003) were cultured on mitomycin C treated STO cell feeder layers in complete medium (Dulbecco's modified Eagle's medium (DMEM)), 10% fetal calf serum, and 10% newborn calf serum) supplemented with 10 ng/ml LIF (Chemicon) and 10 μM beta-mercaptoethanol. These cells express GFP under control of the Olig2 promoter. Undifferentiated ESC were cultured on gelatin-coated dishes for 3-4 passages to remove the feeder cells. For differentiation into ESC-NSC, the feeder cell-free ESC were then plated at 2.5×10⁵ cells per 60 mm dish (gelatin-coated) in N2B27 media (Neurobasal media with 1:50 B27 (Invitrogen) and DMEM/F12 media with N2 supplement (GIBCO/BRL), mixed at 1:1 ratio) for 5 days and the media was refreshed every 2 days. ESC-NSC cells were then passed in the presence of 10 ng/ml bFGF (R&D). Immunofluorescence analysis of markers for NSC showed that during passage ES cells express markers for NSC including BLBP and nestin, which by passage 8 are present in 85% and 90% of cells, respectively (FIG. 6A). These results indicate that GFP-positive (Olig2-positive) cells express both the NSC marker nestin and the radial glial cell marker BLBP.

NSC derived from the G-Olig2 ESC express GFP as a consequence of activation of the Olig2 gene and these cells were purified by FACS at passage 6 (FIG. 6B). GFP positive and negative populations were sorted on a high speed Coulter Epics cell sorter in the Environmental and Occupational Health Science Institute on the Rutgers campus. A histogram of the cell population before FACS sorting shows heterogeneity of GFP intensity and after FACS sorting the histograms shows separation of that GFP-negative and GFP-positive cells (FIG. 6B). The sorted GFP+ and GFP− populations were enriched to 95.81% and 99.85% respectively. Thus FACS can be used to isolate GFP-positive cells expressing both the NSC marker nestin and the radial glial cells marker BLBP.

### Example 9

### Procedure for Immunstaining Cells with Anti-GLAST Monoclonal Antibody at the Cell Surface

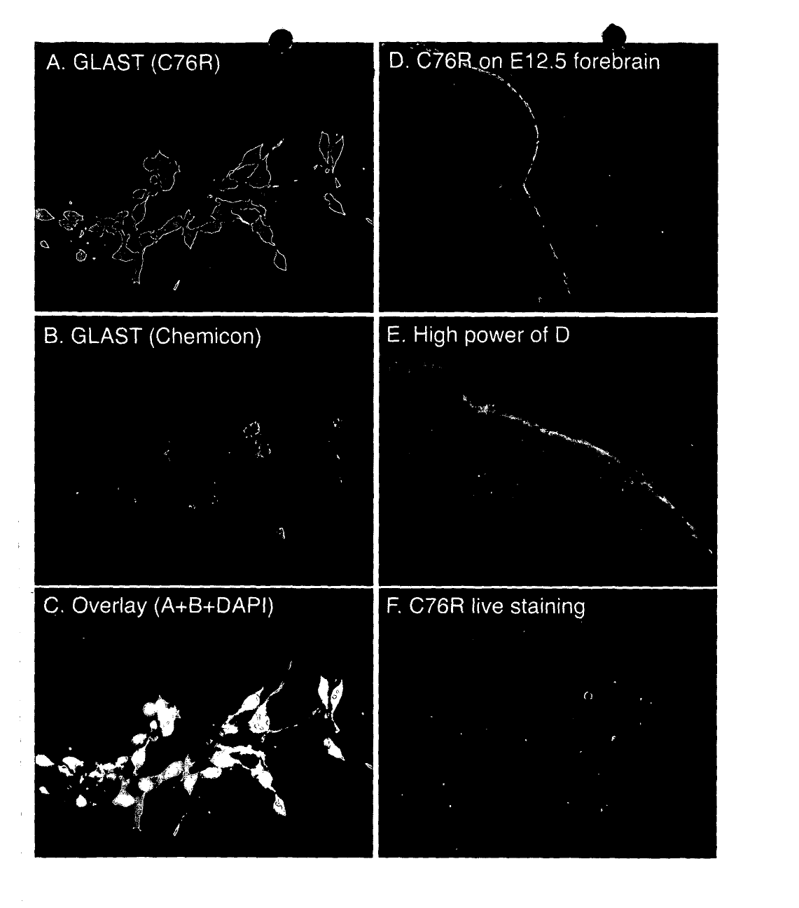

Mouse anti-GLAST antibody (C76R) was used to immunostain embryonic day (E) 15 cortical cells (FIG. 7A-C), E12.5 rat forebrain sections (D,E) and E15 acute cortical cells (F). Cultured E15 cortical cells were fixed with 4% paraformaldehyde for 15 min at room temperature, washed 3 times with phosphate buffered saline (PBS), followed by incubation with primary antibodies mouse anti-GLAST antibody (C76R) and commercial anti-GLAST. Fixed cells were incubated with PBS, 10% normal goat serum including primary antibodies for 1 hour at RT. Triton (0.3%) was included in the staining buffer only when detecting intracellular antigens. After washing with PBS, cells were incubated with secondary antibodies FITC goat-anti-guinea pig IgG (Chemicon) and Rhodamine-Red goat anti-mouse IgG (Molecular Probes), washed and mounted with Gel-Mount (Biomedia corp.).

Mouse anti-GLAST antibody C76R has strong staining on most E15 cortical cells (A) and colocalizes with commercial guinea pig anti-GLAST (Chemicon) (B). Comparison of the distribution of mouse anti-GLAST antibody (C76R) and commercial anti-GLAST in overlays of the images (C) indicated that they bind to the same cells; nuclei were labeled in blue with 10 μg/ml DAPI (Sigma) that was included with the secondary antibody incubations.

For tissue analysis, embryos were dissected from timed pregnant Sprague-Dawley rats (Hilltop), fixed overnight by immersing in 4% paraformaldehyde, cryoprotected with sucrose, and embedded in OCT (Fisher Scientific). Sections of 15 μm were cut, collected on Superfrost Plus microscope slides (Fisher Scientific) and stored at −80° C. Sections were blocked with PBS, 10% normal goat serum for 2 h at RT and then incubated with C76R antibodies for overnight at 4° C. After washing with PBS, secondary Rhodamine-Red goat anti-mouse IgG were applied and incubated for 2 hours at RT. The sections were then washed again and mounted with Gel-Mount.

Mouse anti-GLAST antibody C76R stained E12.5 embryonic cortex strongly in the ventricular zone where the radial glial NSC are located and low background staining was observed on adjacent tissue (D); high power showed cell surface staining pattern in the ventricular zone (E).

When used without paraformaldehyde fixation, mouse anti-GLAST antibody C76R also stains live E15 cortical cells (red); nuclei were labeled with DAPI (blue). The results indicate that mouse anti-GLAST antibody can be useful for live cell staining for FACS.

### REFERENCES

1. Bregman, B. S. Regeneration in the spinal cord. Curr Opin Neurobiol 8, 800-7. (1998).
2. Jones, L. L., Oudega, M., Bunge, M. B. & Tuszynski, M. H. Neurotrophic factors, cellular bridges and gene therapy for spinal cord injury. J Physiol 533, 83-9. (2001).
3. Schwab, M. E. Repairing the injured spinal cord. Science 295, 1029-31 (2002).
4. Gage, F. H. Mammalian neural stem cells. Science 287, 1433-8 (2000).
5. McKay, R. Stem cells in the central nervous system. Science 276, 66-71 (1997).
6. Kim, J. H. et al. Dopamine neurons derived from embryonic stem cells function in an animal model of Parkinson's disease. Nature 418, 50-6 (2002).
7. Hatten, M. E. & Heintz, N. Mechanisms of neural patterning and specification in the developing cerebellum. Annual Review of Neuroscience 18, 385-408 (1995).
8. Norris, C. R. & Kalil, K. Guidance of callosal axons by radial glia in the developing cerebral cortex. J Neurosci 11, 3481-92 (1991).
9. Brittis, P. A., Lemmon, V., Rutishauser, U. & Silver, J. Unique changes of ganglion cell growth cone behavior following cell adhesion molecule perturbations: a time-lapse study of the living retina. Mol Cell Neurosci 6, 433-49 (1995).
10. Gotz, M., Hartfuss, E. & Malatesta, P. Radial glial cells as neuronal precursors: a new perspective on the correlation of morphology and lineage restriction in the developing cerebral cortex of mice. Brain Res Bull 57, 777-88 (2002).
11. Anthony, T., Klein, C., Fishell, G. & Heintz, N. Brain lipid binding protein is a transcriptional target of Notch signalling in neurogenic radial glia. in Society for Neuroscience 352.5 (2003).
12. Noctor, S. C. et al. Dividing precursor cells of the embryonic cortical ventricular zone have morphological and molecular characteristics of radial glia. J Neurosci 22, 3161-73 (2002).
13. Friedlander, D. R. et al. Generation of a radial-like glial cell line. Journal of Neurobiology 37, 291-304 (1998).
14. Hormigo, A., Friedlander, D. R., Brittis, P. A., Zagzag, D. & Grumet, M. Reduced tumorigenicity of rat glioma cells in the brain when mediated by hygromycin phosphotransferase. J Neurosurg 94, 596-604. (2001).
15. Hatten, M. E. The role of migration in central nervous system neuronal development. Current Opinion in Neurobiology 3, 38-44 (1993).
16. Hormigo, A. et al. Integration of embryonic neurons in vivo is enhanced by cotransplantation with a radial glial cell line C6-R. Society for Neuroscience (1999).
17. Hormigo, A. et al. Radial glial cell line C6-R integrates preferentially in adult white matter and facilitates migration of coimplanted neurons in vivo. Exp Neurol 168, 310-22. (2001).
18. Hunter, K. E. & Hatten, M. E. Radial glial cell transformation to astrocytes is bidirectional: Regulation by a diffusible factor in embryonic forebrain. Proceedings of National Academy of Sciences USA 92, 2061-2065 (1995).
19. Li, H., Babiarz, J. & Grumet, M. A Lineage Study on Radial Glia: Relationship with neuronal-restricted precursors (NRPs) and glial-restricted precursors (GRPs). in Sco. For Neurosci. Abs. Vol. 33 564.4 (2003).
20. Villa, A., Snyder, E. Y., Vescovi, A. & Martinez-Serrano, A. Establishment and properties of a growth factor-dependent, perpetual neural stem cell line from the human CNS. Exp Neurol 161, 67-84. (2000).
21. Li, H., Babiarz, J., Jennifer, W., Kane-Goldsmith, N. & Grumet, M. Spatiotemporal Heterogeneity of CNS Radial Glial Cells and Their Transition to Restricted Precursors. Dev Biol, in press (2004).
22. Constantini, S. & Young, W. The effects of methypredniosolone and the ganglioside GM1 on acute spinal cord injury in rats. Journal of Neurosurgery 80, 97-111 (1994).
23. Roonprapunt, C. et al. Soluble cell adhesion molecule L1-Fc promotes locomotor recovery in rats after spinal cord injury. J Neurotrauma 20, 871-82 (2003).
24. Basso, D. M. et al. MASCIS evaluation of open field locomotor scores: effects of experience and teamwork on reliability. Multicenter Animal Spinal Cord Injury Study. J Neurotrauma 13, 343-59 (1996).
25. Basso, D. M., Beattie, M. S. & Bresnahan, J. C. Graded histological and locomotor outcomes after spinal cord contusion using the NYU weight-drop device versus transection. Exp Neurol 139, 244-56 (1996).
26. Takahashi, J., Palmer, T. D. & Gage, F. H. Retionic acid and neurotrophins collaborate to regulate neurogenesis in adult-derived neural tem cell cultures. J. Neurobiol 38, 65-81 (1999).
27. Mabie, P. C. et al. Bone morphogenetic proteins induce astroglial differentiation of oligodendroglial-astroglial progenitor cells. J. Neurosci, 17, 4112-20 (1997).
28. Grumet, M., Flaccus, A. & Margolis, R. U. Functional characterization of chondroitin sulfate proteoglycans of brain: Interactions with neurons and neural cell adhesion molecules. Journal of Cell Biology 120, 815-824 (1993).
29. Grumet, M., Friedlander, D. R. & Sakurai, T. Functions of brain chondroitin sulfate proteoglycans during development: Interactions with adhesion molecules. Perspectives on Developmental Neurobiology 3, 319-330 (1996).
30. Jones, L. L., Yamaguchi, Y., Stallcup, W. B. & Tuszynski, M. H. NG2 is a major chondroitin sulfate proteoglycan produced after spinal cord injury and is expressed by macrophages and oligodendrocyte progenitors. J Neurosci 22, 2792-803 (2002).
31. Davies, S. J. et al. Regeneration of adult axons in white matter tracts of the central nervous system. Nature 390, 680-3 (1997).
32. Arriza, J. L., Fairman, W. A., Wadiche, J. I., Murdoch, G. H., Kavanaugh, M. P., Amara, S. G.; (1994) Functional comparisons of three glutamate transporter subtypes cloned from human motor cortex. J Neurosci 14:5559-5569.
33. Caccamo, D., Katsetos, C. D., Herman, M. M., Frankfurter, A., Collins, V. P., Rubinstein, L. J.; (1989) Immunohistochemistry of a spontaneous murine ovarian teratoma with neuroepithelial differentiation. Neuron-associated beta-tubulin as a marker for primitive neuroepithelium. Lab Invest 60:390-398.
34. Dahlstrand, J., Zimmerman, L. B., McKay, R. D., Lendahl, U.; (1992) Characterization of the human nestin gene reveals a close evolutionary relationship to neurofilaments. J Cell Sci 103 (Pt 2):589-597.
35. Dietrich, J., Noble, M., Mayer-Proschel, M.; (2002) Characterization of A2B5+ glial precursor cells from cryopreserved human fetal brain progenitor cells. Glia 40:65-77.
36. Draper, J. S., Pigott, C., Thomson, J. A., Andrews, P. W.; (2002) Surface antigens of human embryonic stem cells: changes upon differentiation in culture. J Anat 200:249-258.
37. Eisenbarth, G. S., Walsh, F. S., Nirenberg, M.; (1979) Monoclonal antibody to a plasma membrane antigen of neurons. Proc Natl Acad Sci USA 76:4913-4917.
38. Feng, L., Heintz, N.; (1995) Differentiating neurons activate transcription of the brain lipid-binding protein gene in radial glia through a novel regulatory element. Development 121:1719-1730.
39. Hockfield, S., McKay, R.; (1985) Identification of major cell classes in the developing mammalian nervous system. J Neurosci 5:3310-3328.
40. Kawakami, H., Tanaka, K., Nakayama, T., Inoue, K., Nakamura, S.; (1994) Cloning and expression of a human glutamate transporter. Biochem Biophys Res Commun 199:171-176.
41. Li, H., Babiarz, J., Woodbury, J., Kane-Goldsmith, N., Grumet, M.; (2004) Spatiotemporal heterogeneity of CNS radial glial cells and their transition to restricted precursors. Dev Biol 271:225-238.
42. Liu, Y., Wu, Y., Lee, J. C., Xue, H., Pevny, L. H., Kaprielian, Z., Rao, M. S.; (2002) Oligodendrocyte and astrocyte development in rodents: an in situ and immunohistological analysis during embryonic development. Glia 40:25-43.
43. Quinn, S. M., Walters, W. M., Vescovi, A. L., Whittemore, S. R.; (1999) Lineage restriction of neuroepithelial precursor cells from fetal human spinal cord. J Neurosci Res 57:590-602.
44. Shibata, T., Yamada, K., Watanabe, M., Ikenaka, K., Wada, K., Tanaka, K., Inoue, Y.; (1997) Glutamate transporter GLAST is expressed in the radial glia-astrocyte lineage of developing mouse spinal cord. J Neurosci 17:9212-9219.
45. Marchal-Victortian, S., et al. ; (2003) The Human NTERA2 neural cell line generates neurons on growth under neural stem cell conditions and exhibits characteristics of radial glia cell S. Molecular and cellular Neurosciences 24: 198-213.
46. Hasegawa, K. and Grumet, M.; (2003) Trauma induces tumorigenesis of cells implanted into rat spinal cord; J. Neurosurgery 98:1065-1071.
47. Xian, H. Q., McNichols, E., St Clair, A., and Gottlieb, D. I. (2003). A subset of ES-cell-derived neural cells marked by gene targeting. Stem Cells 21, 41-9.

What is claimed is:

1. An enriched population of radial glial cells, wherein the cells have the following characteristics:
nestin⁺,
BLBP⁺,
non-tumorigenic, and
capable of:
exhibiting bipolar morphology,
self-renewing,
migrating and self-organizing in white matter, and
supporting neuronal migration.

2. The cells of claim 1, wherein the cells are GLAST⁺ or derived from a cell that is GLAST⁺.

3. The cells of claim 1, wherein the cells are TuJ1⁻.

4. The cells of claim 1, wherein the cells are SSEA4⁻.

5. The cells of claim 1, wherein the cells are inhibited from differentiating into neurons, oligodendrocytes and astrocytes in vitro.

6. The cells of claim 5, wherein the cells have been modified to express an oncogene.

7. The cells of claim 5, wherein the cells have increased expression levels of the Notch protein or an intracellular derivative thereof.

8. The cells of claim 5, wherein the cells have increased expression levels of the BLBP protein.

9. The cells of claim 5, wherein the cells have increased expression levels of glial growth factor erbB2/receptor.

10. The cells of claim 9, wherein the glial growth factor erbB2/receptor is activated.

11. The cells of claim 1, wherein the cells are capable of giving rise to neurons in vitro.

12. The cells of claim 1, wherein the cells are derived from adult stem cells.

13. The cells of claim 1, wherein the cells are derived from embryonic stem cells.

14. The cells of claim 1, wherein the cells are derived from neural stem cells.

15. The cells of claim 1, wherein the cells are derived from neurospheres.

16. The cells of claim 1, wherein the cells are A2B5⁻.

17. A method for protecting neural tissue in a human patient in need thereof, the method comprising administering to the patient a therapeutically effective amount of an enriched population of cells according to claim 1.

18. The method of claim 17, wherein the cells are administered to a patient that has a neural injury.

19. The method of claim 18, wherein the neural injury is selected from the group consisting of spinal cord injury and stroke.

20. The method of claim 17, wherein the cells are administered to a patient that has a neurodegenerative disease.

21. The method of claim 20, wherein the neurodegenerative disease is selected from the group consisting of Multiple Sclerosis, Amyotrophic Lateral Sclerosis, Parkinson's disease, Huntington's Disease and Alzheimer's Disease.

22. The method of claim 17, wherein the cells are injected intrathecally into a neural injury site.

23. The method of claim 17, wherein the cells are injected intrathecally adjacent to a neural injury site.

24. The method of claim 17, wherein the cells are injected intrathecally into a neurodegenerative disease site.

25. The method of claim 17, wherein the cells are injected intrathecally adjacent to a neurodegenerative disease site.

26. The method of claim 17, wherein the cells are introduced intravenously.

27. The method of claim 17, wherein the cells are introduced intraventricularly.

28. A method for promoting axonal regrowth in a human patient in need thereof, the method comprising administering to the patient a therapeutically effective amount of an enriched population of cells according to claim 1.

29. The method of claim 28, wherein the cells are administered to a patient that has a neural injury.

30. The method of claim 29, wherein the neural injury is selected from the group consisting of spinal cord injury and stroke.

31. The method of claim 30, wherein the cells enhance the organization of spinal cord tissue following the injury.

32. The method of claim 30, wherein the cells suppress the deposition of chondroitin sulfate proteoglycans in the spinal cord tissue following the injury.

33. The method of claim 30, wherein the cells suppress the infiltration of macerophages in the spinal cord tissue following the injury.

34. The method of claim 28, wherein the cells are administered to a patient that has a neurodegenerative disease.

35. The method of claim 34, wherein the neurodegenerative disease is selected from the group consisting of Multiple Sclerosis, Amyotrophic Lateral Sclerosis, Parkinson's disease, Huntington's Disease and Alzheimer's Disease.

36. The method of claim 28, wherein the cells are injected intrathecally into a neural injury site.

37. The method of claim 28, wherein the cells are injected intrathecally adjacent to a neural injury site.

38. The method of claim 28, wherein the cells are injected intrathecally into a neurodegenerative disease site.

39. The method of claim 28, wherein the cells are injected intrathecally adjacent to a neurodegenerative disease site.

40. The method of claim 28, wherein the cells are administered intravenously.

41. The method of claim 28, wherein the cells are administered intraventricularly.

42. The method of claim 28, wherein the axonal regrowth comprises sprouting.

43. The method of claim 28, wherein the axonal regrowth comprises regeneration.

44. A method for enriching a population of cells according to claim 1, the method comprising separating from a mixed population of cells the cells according to claim 1.

45. The method of claim 44, wherein the mixed population of cells are contacted with a molecule that binds specifically to a marker on the cells according to claim 1; and the bound cells are isolated from unbound cells.

46. The method of claim 45, wherein the bound cells are separated from the molecule.

47. The method of claim 44, wherein the selected cells are GLAST⁺ or are derived from GLAST⁺ cells.

48. The method of claim 47, wherein the GLAST⁺ cells are contacted with a molecule that binds specifically to GLAST.

49. The method of claim 48, wherein the molecule is an antibody.

50. The method of claim 49, wherein the antibody is labeled.

51. The method of claim 50, wherein the antibody is fluorescently-labeled.

52. The method of claim 44, wherein the mixed population of cells comprises a reporter indicating transcription of the gene for a marker associated with the cells according to claim 1.

53. The method of claim 52, wherein the marker comprises a protein.

54. The method of claim 52, wherein the marker is selected from the group consisting of BLBP, GLAST and nestin.

55. The method of claim 44, wherein the mixed cell population is transfected with a DNA molecule comprising a reporter gene operatively linked to a promoter for a marker gene associated with the cells according to claim 1.

56. The method of claim 55, wherein the marker is selected from the group consisting of BLBP, GLAST and nestin.

57. A method for isolating a GLAST⁺ radial glial cell population comprising:
providing a mixed population of GLAST⁺ and GLAST⁻ cells;
binding the cells to an antibody that recognizes an extracellular portion of GLAST; and
isolating the antibody-bound cells from unbound cells.

58. The method of claim 57, further comprising separating the cells from the antibody.

59. The method of claim 57, wherein the radial glial cell population is isolated from neural stem cells.

60. The method of claim 57, wherein the radial glial cell population is isolated from adult neural stem cells.

61. The method of claim 57, wherein the radial glial cell population is isolated from embryonic stem cells.

62. The method of claim 57, further comprising isolating GLAST⁺ cells or an enriched population of GLAST⁺ cells.

63. A monoclonal antibody that specifically recognizes an extracellular portion of GLAST.

64. The monoclonal antibody of claim 63, wherein the antibody is raised against human GLAST.

65. The monoclonal antibody of claim 63, wherein the antibody specifically recognizes the region of GLAST corresponding to about amino acid 65 to about amino acid 82 of human GLAST (SEQ ID NO: 1).


SEQUENCE LISTING (1 sequence)

SEQ ID NO: 1 - protein, 542 aa, homo sapiens
MTKSNGEEPKMGGRMERFQQGVRKRTLLAKKKVQNITKEDVKSYLFRNAFVLLTVTAVIVGTILGFTLRPYRMSYREVKYFSFPGELLMRMLQMLVLPLIISSLVTGMAALDSKASGKMGMRAVVYYMTTTIIAVVIGIIIVIIIHPGKGTKENMHREGKIVRVTAADAFLDLIRNMFPPNLVEACFKQFKTNYEKRSFKVPIQANETLVGAVINNVSEAMETLTRITEELVPVPGSVNGVNALGLVVFSMCFGFVIGNMKEQGQALREFFDSLNEAIMRLVAVIMWYAPVGILFLIAGKIVEMEDMGVIGGQLAMYTVTVIVGLLIHAVIVLPLLYFLVTRKNPWVFIGGLLQALITALGTSSSSATLPITFKCLEENNGVDKRVTRFVLPVGATINMDGTALYEALAAIFIAQVNNFELNFGQIITISITATAASIGAAGIPQAGLVTMVIVLTSVGLPTDDITLIIAVDWFLDRLRTTTNVLGDSLGAGIVEHLSRHELKNRDVEMGNSVIEENEMKKPYQLIAQDNETEKPIDSETKM